(19) United States
(12) Patent Application Publication
(10) Pub. No.: US 2011/0276344 A1
(43) Pub. Date: Nov. 10, 2011

(54) METHOD FOR DIAGNOSING FOOD ALLERGY

(76) Inventor: Paul Eirian Williams, South Glamorgan (GB)

(21) Appl. No.: 13/144,548

(22) PCT Filed: Jan. 14, 2010
(86) PCT No.: PCT/GB2010/050050

§ 371 Date: Jul. 14, 2011

(30) Foreign Application Priority Data
Jan. 15, 2009 (GB) 0900623.0

(51) Int. Cl. G06Q 50/00; G06N 3/02; A61K 49/00

(52) U.S. Cl. 705/2; 424/9.81; 706/20

(57) ABSTRACT

According to the invention there is provided a method for diagnosing food allergy including: (a) asking a patient each of the following questions: are any drugs which can cause the symptoms complained of being taken or have recently been taken; are symptoms triggered by nuts other than peanut; are symptoms triggered by a specific food other than a nut; are symptoms triggered by fruit and vegetables; (b) carrying out each of the following tests: skin prick test to a plurality of nuts to determine if there is a reactivity to any one of them; RAST test to a plurality of nuts in order to determine the highest quantitative result; and (c) inputting the results of the questions and tests into a neural network that has been trained to diagnose food allergy, wherein the highest quantitative result from the RAST test to a plurality of nuts is inputted; and (d) producing an output indicative of a food allergy.

### FIELD OF THE INVENTION

This invention relates to a method and means, including parts thereof, for diagnosing food allergy, using an artificial neural network (ANN). The invention involves obtaining information about a patient, based on asking the patient a series of selected questions and carrying out a number of selected tests, inputting this information into a neural network, and obtaining a preliminary diagnosis. The invention applies equally to adults and children.

### BACKGROUND OF THE INVENTION

Allergies currently affect approximately 34% of the general population (Linneberg 2000). Whilst at one extreme serious conditions such as anaphylaxis can be life threatening, most allergic disorders pose little risk of death. However, diseases such as food allergy cause distress and misery for millions of patents, often at times in their lives when they should be most active (Holgate and Broide 2003). Allergic diseases are a significant cause of morbidity in modern society, adversely affecting sleep, intellectual functioning and recreational activities; food allergy may lead to considerable anxieties for fear of inadvertently ingesting the offending allergen (Holgate 1999). Furthermore, allergic diseases exert a profoundly negative impact on occupational performance and have major public health costs.

Across the United Kingdom, waiting times for specialist allergy consultations following referral from primary care are long.

The rising prevalence of allergies and the associated demand for specialist services suggest that waiting times will inevitably lengthen over the course of the next decade. Given that there is currently an acute shortage of Immunologists and Allergists in the UK and worldwide, it seems unlikely that sufficient medical manpower will emerge in the foreseeable future to deal with this increasing demand.

Recent in-house research has centred on the role of the Allergy Nurse Practitioner in the diagnosis and management of allergic disease. Increasing use of the Nurse Practitioner in a diagnostic role would enable waiting times to be shortened and new patient referrals to be seen without the presence of the Consultant Clinical Immunologist. Whilst Nurse Practitioner-based diagnosis and management strategies should, in time, ameliorate the critical situation, a parallel increase in demand for allergy services will, without doubt, limit the positive effects on waiting times. There therefore remains a need to develop further innovative methods to facilitate access of patients to clinical diagnostic services.

However, as one would expect, it is extremely important that any new methods of diagnosis are accurate if they are to be adopted by the medical community at large. These methods must be able to replicate, it not exceed, the accuracy of an experienced Clinical Immunologist. This is a difficult task to achieve because a Clinical Immunologist uses information from a vast number of sources when reaching a diagnosis.

Typically, when diagnosing a condition, a medical practitioner will integrate information from several sources, such as a medical history, a physical examination, the results of clinical tests, and by asking the patient about his/her condition. The medical practitioner will use judgement based on experience and intuition, both when deciding what to look for and in analysing the information, in order to come to a particular diagnosis.

Thus, the process of diagnosis involves a combination of knowledge, intuition and experience that leads a medical practitioner to ask certain questions and carry out particular clinical tests, and the validity of the diagnosis is very dependent upon these factors.

Given the predictive and intuitive nature of medical diagnosis, and the fact that specialist, experienced medical practitioners are in demand, we have attempted to replicate the diagnostic process in an automated system, in order to give a wider audience access to this service. We have found that artificial neural networks (ANNs) have characteristics that make them particularly well suited for this purpose.

ANNs are computational mathematical modelling tools for information processing and may be defined as ‘structures comprised of densely interconnected adaptive processing elements (nodes) that are capable of performing massively parallel computations for data processing and knowledge representation’ (Hecht-Nielsen 1990; Schalkoff 1977). Single artificial neurons for the computation of arithmetic and logical functions were first described by McCulloh and Pitts (1943); fifteen years later Rosenblatt (1958) described the first successful neurocomputer (the Mark 1 Perceptron). This simple network consisted of two layers of neurons connected by a single layer of weighted links and was capable of solving problems in a way analogous to information processing in the human brain (Wei et al 1998; Basheer and Hajmeer 2000). These early structures were however unable to predict generalised solutions for complex non-linear problems. Over the course of the following five decades complexity has increased with the development of multiple networked perceptrons; such advances have led to the application of ANNs to a colossal number of problems, and by 1994 more than 50 different types of network were in existence (Pham 1994 and Basheer and Hajmeer 2000), each possessing unique properties enabling them to solve particular tasks.

Such ANNs are capable of dealing with non-linear data, fault and failure, high parallelism and imprecise and fuzzy information (Wei et al 1998). Neural networks have been shown to be capable of modelling complex real-world problems and found extensive acceptance in many scientific disciplines (Callan 1999). The decision as to which type of ANN should be utilised for a particular task depends on problem logistics, input type, and the execution speed of the trained network (Basheer and Hajmeer 2000).

Neural networks have found increasing application in a range of clinical settings where they have produced accurate and generalised solutions compared to traditional statistical methodology (reviewed Baxt 1995, Wei et al 1998, Dybowski and Gant 2001). For example, U.S. Pat. No. 6,678,669 discloses using an ANN to diagnose endometriosis, predicting pregnancy related events, such as the likelihood of delivery within a particular time period, and other such disorders relevant to women's health.

The most commonly used ANN in such studies is the Backpropagational Multilayer Perceptron (MLP). MLPs are particularly useful in solving pattern classification problems (Wei et al 1998; Basheer and Hajmeer, 2000), which are common in the clinical arena. In this context the ANN looks for patterns in a similar way to learning in the human mind; the more a particular pattern is represented, the stronger the recognition of it by the network.

We have developed a method of diagnosing food allergy using a neural network. In particular, from the vast amount of information that a clinician would have available, we have identified a manageable set of questions and tests that have clinical significance, and can be used to train a neural network to diagnose food allergy, and by inputting the results of these questions and tests into a neural network thus trained the network to produce a diagnosis.

Surprisingly, we have found that a small set of just 6 inputs to the neural network have a profound influence on the provision of an accurate diagnosis.

We have also identified a set of 19, 22, 27, 32, 40, 47, 60 and 79 inputs, referred to in this description as the 19, 22, 27, 32, 40, 47, 60 and 79 input models respectively, that can be input into a neural network to obtain a diagnosis.

The identification of these clinically significant questions and tests will mean that a neural network can be trained to diagnose food allergy in considerably less time than it currently takes a consultant, which in turn will save time and money.

Additionally, a neural network offers an easy-to-use means of diagnosis, both for clinicians and non-clinicians, and will allow central aspects of diagnosis and management to be performed electronically in a way that is accessible to systematic audit and reduce inequalities in accessing allergy services, via the use of remote electronic information transfer.

For the avoidance of doubt, any reference herein to a neural network is a reference to an artificial neural network (ANN).

According to a broad aspect of the invention, there is provided a method for diagnosing food allergy asking a patient a set of questions and/or carrying out one or more tests; inputting the results of the questions and tests into a neural network that has been trained to diagnose food allergy; and producing an output indicative of food allergy.

According to a first aspect of the invention, there is therefore provided a method for diagnosing food allergy including:
(a) asking a patient each of the following questions:
   are any drugs which can cause the symptoms complained of being taken or have recently been taken;
   are symptoms triggered by nuts other than peanut;
   are symptoms triggered by a specific food other than a nut;
   are symptoms triggered by fruit and vegetables;
(b) carrying out each of the following tests:
   skin prick test (SPT) to a plurality of nuts to determine if there is a reactivity to any one of them;
   RAST test to a plurality of nuts in order to determine the highest quantitative result; and
(c) inputting the results of the questions and tests into a neural network that has been trained to diagnose food allergy, wherein the highest quantitative result from the RAST test to a plurality of nuts is inputted; and
(d) producing an output indicative of a food allergy.

This is referred to as the 6-input model.

Typically, the RAST and SPT tests to a plurality of nuts includes tests on peanut, hazelnut, almond, walnut and brazil nut. Other selections of nuts may suggest themselves to the skilled person.

In a preferred method of the invention,
part (a) further includes asking the patient the following questions:
is tingling of the mouth or lips, swelling of the tongue, the inside of the mouth or throat, difficulty swallowing, or difficulty breathing experienced after foods;
are symptoms triggered by wheat;
are symptoms triggered by milk;
are symptoms triggered by peanut;
are symptoms triggered by shellfish;
the time elapsed since eating a food implicated with causing symptoms and the symptoms appearing;
how frequently the symptoms occur;
length of time that rash or swelling has been experienced;
and part (b) further includes carrying out the following tests:
skin prick test to grass pollens;
RAST test to milk;
RAST test to fish;
one or more RAST tests to any fruit, vegetable or other food (other than egg, milk, soya, wheat, fish, rice, peanut, hazelnut, brazil nut, almond, walnut or apple) associated with symptoms;
RAST test to any specific food other than nuts associated with the symptoms.

This is referred to as a 19-input model.

In yet a further preferred method of the invention:
part (b) further includes carrying out the following tests:
RAST test to grass pollens;
RAST test to fish;
RAST test to apple.

This is referred to as a 22-input model.

In yet a further preferred method of the invention:
part (a) further includes asking the patient the following questions:
is nausea, vomiting, abdominal pain or diarrhea experienced after foods;
is wheezing or a worsening of asthma or eczema experienced after eating foods;
are symptoms triggered by cheese;
what areas of the body are affected by a rash;
and part (b) further includes carrying out the following tests:
RAST test to cat.

This is referred to as a 27-input model.

In yet a further preferred method of the invention:
part (a) further includes asking the patient the following questions:
number of first degree relatives with asthma, rhinitis or eczema;
is a nettle rash experienced after foods;
and part (b) further includes carrying out the following tests:
skin prick test to hazelnut;
skin prick test to walnut;
RAST test to rice.

This is referred to as a 32-input model.

In yet a further preferred method of the invention:
part (b) further includes carrying out the following tests:
skin prick test to HDM (house dust mite);
skin prick test to peanut;
skin prick test to brazil nut;
skin prick test to almond;
RAST test to HDM;
RAST test to dog;
RAST test to peanut;
RAST test to brazil nut.

This is referred to as a 40-input model.

In yet a further preferred method of the invention:
part (a) further includes asking the patient the following questions;
are headaches experienced after foods;
are symptoms triggered by aspirin, aspirin-containing drugs, orange juice, curry, or high aspirin content food;
are antihistamines effective;
and part (b) further includes carrying out the following tests:
skin prick test for dog;
RAST test to egg;
RAST test to almond;
RAST test to walnut.

This is referred to as a 47-input model.

In yet a further preferred method of the invention:
part (a) further includes asking the patient the following questions:
number of pack years smoked;
are symptoms triggered by egg;
are symptoms triggered by fish;
are symptoms triggered by unidentified food additives;
and part (b) further includes carrying out the following tests:
skin prick test to cat;
skin prick test to tree pollens;
skin prick test to egg;
skin prick test to milk;
skin prick test to rice;
total serum (IgE) detected;
RAST test to tree pollens;
RAST test to soya;
RAST test to hazelnut.

This is referred to as a 60-input model.

In yet a further preferred method of the invention:
part (a) further includes asking the patient the following questions:
is an ACE (Angiotensin Converting Enzyme) inhibitor being taken;
is an A2R (Angiotensin-2 receptor) antagonist being taken;
is a statin being taken;
is a PPI (Proton Pump Inhibitor) being taken;
is a SSRI (Selective Serotonin Reuptake Inhibitor) being taken;
is SNRI (Serotonin and Noradrenalin Reuptake Inhibitor) being taken;
are any NSAIDs (Non-Steroidal Anti-Inflammatory Drugs) or aspirin being taken;
is OCPill (Oral Contraceptive Pill) being taken;
is HRT (Hormone Replacement Therapy) being taken;
is a bisphosphonate being taken;
are any other drugs that are associated with urticaria or angioedema being taken;
is tingling of the mouth or lips, swelling of the tongue, the inside of the mouth or throat, difficulty swallowing or difficulty breathing experienced after other medications than those known to cause urticaria or angioedema;
is swelling of the lips, eyelids or tongue experienced;
is an itchy, red, raised, burning and hot nettle rash experienced;
how long do new rash patches appear for;
do rash patches last for;
do symptoms come on with physical stimuli such as cold, wet, wind and pressure;
and part (b) further includes carrying out the following tests:
SPT test to latex;
RAST test to latex.

This is referred to as a 79-input model.

The question ‘how long do rash patches last for’ may be coded for a yes/no answer; for example, whether the patches last for longer or shorter than a defined period of time, such as 24 hours.

Examples of drugs that are associated with urticaria or angioedema include opiates, nicorandil, amlodipine, X-ray contrast media and chlorthalidone. Other examples are known to the skilled person. Drugs causing gastrointestinal symptoms include ACE Inhibitors, Statins, Proton Pump inhibitors, Selective Serotonin Re-uptake Inhibitors, Serotonin and Noradrenaline Reuptake Inhibitors, Bisphosphonates and Opiates. Other examples are known to the skilled person.

Preferably, food allergy is diagnosed according to aetiological cause. Advantageously, nut allergy may be diagnosed by the neural network.

At least one of food intolerance, drug-induced multiple allergy (oral allergy syndrome, pollen allergy and/or nut allergy) and allergy to other foods may be diagnosed by the neural network.

Generally, the results of the tests under part (b) are provided as quantitative results. The quantitative results may relate to the amount of allergen-specific IgE antibodies present. The results of the tests under part (b) above may be provided with a graded result and so represent an incremental unit indicative of the nature of the response. Alternatively, the results may represent a measure of a unit from a continuous scale, such as kilo units of allergen-specific IgE antibodies per litre.

Grass and tree pollens referred to herein may be selected having regard to the geographical region in which the patient lives. For example, in the UK, one would test for mixed grass pollens whereas in North America one is much more likely to include ragweed and in Northern Europe a test for tree pollen is likely to include a test for tree birch. As will be apparent to the man skilled in the art the geographically representative allergens are well known in each geographical region and would be selected on the basis that in each region the selected allergens are known to elicit an allergic reaction of the upper respiratory tract.

The RAST test is undertaken using an antibody that is labelled with a suitable label such as a radio-label, although light emitting labels may be used as an alternative, and conventional techniques are used in order to measure the patient's immune status. RAST tests, and variations thereof, are well known to those skilled in the art and indeed have been performed for many decades. The original disclosure concerning diagnosis of an allergy by an in vitro test for allergen antibodies was described by Wide et al in 1967 and has further been assessed by Thomson & Bird, 1983.

In some cases it may be useful to save results for analysis at a later time, for example if they cannot be obtained simultaneously. In this instance the results may be stored on a computer system and applied to a neural network subsequently.

In another aspect of the invention, there is provided a computer system or apparatus, configured to aid in the diagnosis of food allergy including:
(a) a device for obtaining data relating to a patient, wherein the data includes the results of a combination of questions and tests outlined in the first aspect of the invention;
(b) optionally, a device for storing the data in storage means of the computer system;
(c) a device for transferring the data to a neural network trained on samples of the data; and
(d) a device for extracting from the trained neural network an output, the output being an indicator for the diagnosis of food allergy.

For the avoidance of doubt, in the computer system or apparatus the data comprises information obtained using the 6-, 19-, 22-, 27-, 32-, 40-, 47-, 60- or 79-input model, or any selected combination thereof.

As will be appreciated, this aspect of the invention may also be adapted so that the computer is linked to an intranet or Internet with a neural network, thereby allowing patients and/or medical practitioners to input information from remote locations and obtain a preliminary diagnosis.

According to a further aspect of the invention there is provided a neural network to aid in the diagnosis of food allergy, the neural network including:
an input layer having a plurality of input nodes into which can be inputted data which include the results of an combination of questions and tests outlined in the first aspect of the invention; and
an output layer for producing an output;
in which the neural network is trained on data relating to a group of patients in which food allergy is present, wherein the data includes said results of said combination of questions and tests outlined in the first aspect of the invention, so that the neural network is configured to identify a pattern of data which corresponds to food allergy by the output layer producing an output indicative of the diagnosis of food allergy.

The results of any of the 6-, 19-, 22-, 27-, 32-, 40-, 47-, 60-, or 79-input models, or any selected combination thereof, may also be used to train a neural network to diagnose a condition.

Accordingly, in a further aspect of the invention there is provided a method for training a neural network to aid in diagnosing food allergy, including:
a) obtaining data relating to a group of patients in which food allergy is known, wherein the data include a combination of the results of the questions and tests outlined in the first aspect of the invention;
(b) training a neural network to identify a pattern of data which corresponds to food allergy; and
(c) storing the neural network in storage means of a computer or on a computer-readable medium.

A neural network may also be trained using other methods, which methods will be apparent to a man skilled in the art.

The invention further comprises a computer or a computer system comprising at least one neural network embodying any one or more of the aforementioned models or methods for the purposes of performing a diagnosis.

The invention further comprises at least one neural network that has been trained for diagnosis using data from the 6-, 19-, 22-, 27-, 32-, 40-, 47-, 60- or 79-input models. Such a neural network may be sold separately, or put on a server so that it can be accessed remotely.

Yet further, the invention comprises a data carrier comprising the aforementioned methodology of the invention and/or a software interface for enabling a user to communicate with a neural network trained for the diagnostic purpose of the invention.

According to another aspect of the present invention there is provided a computer program product including:
a computer usable medium having computer readable program code and computer readable system code embodied on said medium for aiding in the diagnosis of a food allergy, said computer program product including:
computer program code means, when the program code is loaded, to make the computer execute a procedure to:
(a) obtain data relating to a patient, wherein the data includes the results of a combination of questions and tests outlined in the first aspect of the invention;
(b) optionally, store the data;
(c) transfer the data to a neural network trained on the aforementioned data; and
(d) extract from the trained neural network an output, the output being an indicator for the diagnosis of food allergy.

According to a further aspect of the invention there is provided a computer system including a first means for:
(a) obtaining data relating to a patient, wherein the data includes the results of a combination of questions and tests outlined in the first aspect of the invention; and
   a second remote means, wherein said second means includes means for:
(b) optionally, storing the data;
(c) transferring the data to a neural network trained on the aforementioned data; and
(d) extracting from the trained neural network on output, the output being an indicator for the diagnosis of food allergy.

Whilst the invention has been described above, it extends to any inventive combination of the features set out above, or in the following description, drawings or claims.

The present invention will now be illustrated with reference to the following method and results.

### EXAMPLE 1

Table 1 shows the distribution of diagnoses in patients presenting to the Welsh Clinical Allergy Service (WCAS) outpatient clinics in 2001, and is representative of the caseload seen in this regional allergy centre. It will be seen that a significant proportion of patients presenting to the service with symptoms of food allergy (including food intolerance).

TABLE 1
Distribution of diagnoses in patients seen in
WCAS outpatient clinics in 2001 (n = 213).

| | No. of patients | Percentage of all |
|---|---|---|
| Diagnostic Category | with diagnosis | patients (%) |
| Urticaria/Angioedema | 46 | 21.6 |
| Rhinitis | 43 | 20.2 |
| Drug-induced angioedema/reaction | 28 | 13.1 |
| Food allergy | 26 | 12.2 |
| Food intolerance | 14 | 6.6 |
| Salicylate intolerance | 11 | 5.2 |
| Venom insensitivity | 7 | 3.3 |
| Non-allergic/ | 38 | 17.8 |
| miscellaneous conditions | | |
| Total | 213 | 100 |

### Methods

### Ethical Considerations

Bro Taf Local Research Ethics Committee granted ethical approval for all aspects of this study and the project was registered with Cardiff and Vale NHS Trust Research and Development Office. All participants were required to complete a consent form. Data were anonymised prior to analysis and handled in accordance with the Data Protection Act 1998.

### Structured Questionnaire Design

This study made use of a standard questionnaire comprising questions and tests, which was created using the commercial Cardiff TELEform information capture system v7.0 Designer module. This questionnaire was devised as an integral part of the Nurse Practitioner-based diagnosis and management evaluation program and aimed to gather demographic and clinical information in a structured format. This questionnaire was endorsed by a multidisciplinary panel of experts and piloted in WCAS clinics throughout 2001.

### Patient Recruitment and Data Collection

Data were gathered during 2004. Patients aged 18 to 75 referred to the WCAS by General Practitioners or hospital doctors due to symptoms of food allergy were drawn from the routine non-urgent outpatient waiting list and recruited using an approved protocol. All consenting patients with predominant presenting symptoms of food allergy were entered into the study. There were no exclusion criteria. Participants underwent Skin Prick Testing immediately prior to an initial conventional consultation with either the Consultant Clinical Immunologist or Allergy Nurse Practitioner. The order of consultation was randomized so that roughly equal numbers of patients were seen first by the Nurse Practitioner as by the Consultation Clinical Immunologist. Findings were recorded on the standard questionnaire ensuring all sections were fully completed. Patients were then seen independently by the other practitioner, and findings annotated upon a separate questionnaire. Total serum IgE and RAST testing were performed upon clinical discretion. As per current WCAS protocol, a clinic letter outlining the final diagnosis and management plan was dictated by the Consultant Clinical Immunologist and posted to the referring medical practitioner and patient. A similar letter was dictated independently by the Allergy Nurse Practitioner, which was retained as supporting evidence to her questionnaire, for analysis in a later study.

### Data Transfer

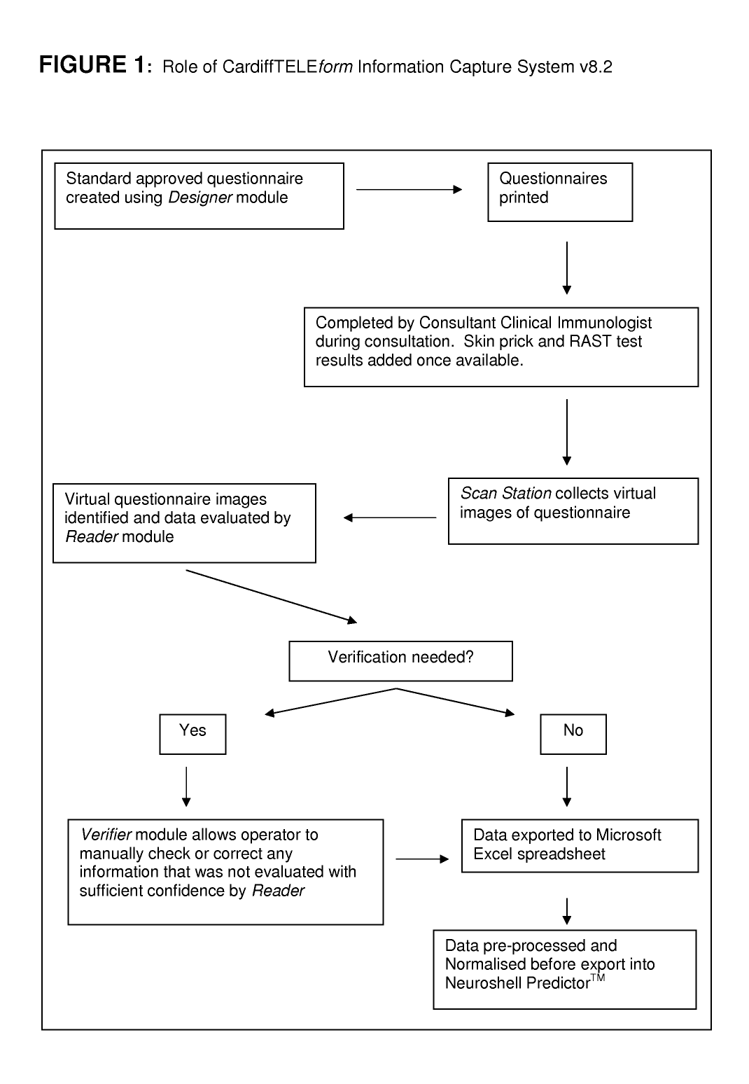

Once available, all RAST and other test results were added to data recorded during respective consultations. Completed questionnaires were processed using the commercial Cardiff TELEform information capture system v8.2 Scan station, Reader and Verifier modules (see FIG. 1). Data were exported into separate Microsoft Excel files for each clinician.

### Data Preprocessing and Normalisation

Data imported into Microsoft Excel were anonymised. All input variables were inspected for transfer accuracy and errors corrected manually. Data were normalised (scaled) within a uniform range for each input variable, some variables removed (e.g. domestic demographic data, ethnic origin and marital status) and a number of new input variables created following recoding of defined input groups. The final aetiological diagnosis for each patient was coded into one of five output categories (food intolerance, nut allergy, drug-induced GI symptoms, multiple allergy, and allergy to a specific food (other than nut)). The multiple allergy category covers oral allergy syndrome, pollen allergy and nut allergy, and any combination of these causes.

### Data Partitioning

Data were partitioned into two separate Excel parent databases (i.e. separate Excel worksheets) (i) ‘all questionnaire inputs’ and (ii) ‘clinically selected inputs’ (79 input variables; five output variables) (see Table 2), as it became available. ANN models were developed using data and diagnoses from the Consultant Clinical Immunologist. Model development required data from each parent database to be divided into two subsets: (i) training and test data and (ii) validation.

TABLE 2
Questions and test results utilised in 79-, 60-, 47-, 40-, 32-, 27-, 22-,
19- and 6-input models.
Inputs used in different analyses
No of inputs used: 79 60 47 40 32 27 22 19 13

Taking ACE inhibitor
Taking A2R antagonist
Taking Statin
Taking PPI
Taking SSRI
Taking SNRI
Taking NSAID or Aspirin
Taking OCPIII
Taking HRT
Taking Bisphosphonates
Given other drugs associated with angioedema and urticaria (opiates,
nicorandil, amlodipine, contrast media, chlorthalidone)
Taking any drugs that can cause those symptoms complained of (urticaria,
angioedema etc.. abdominal symptoms etc . . . )
Tingling of the mouth/lips, swelling of the tongue, inside mouth, throat or
difficulty swallowing or breathing after other medications than those
known to cause urticaria or angioedema?
No of pack-years smoked
No of first degree relatives with asthma, rhintis, eczema
Tingling of the mouth/lips, swelling of the tongue, inside mouth, throat or
difficulty swallowing or breathing after foods
Nausea, vomiting, abdominal pain or diarrhoea after foods
Nettle rash after foods
Wheeze, worsening of asthma or eczema after eating foods
Headaches after foods
Symptoms triggered by wheat
Symptoms triggered by egg
Symptoms triggered by milk
Symptoms triggered by cheese
Symptoms triggered by peanut
Symptoms triggered by other nuts than peanut
Symptoms triggered by fish
Symptoms triggered by shellfish
Symptoms triggered by specific food other than a nut
Symptoms triggered by fruit & vegetables
Symptoms triggered by unidentified food additives
Symptoms triggered by Aspirin, aspirin-containing drugs, orange juice,
curry or high-aspirin content food
No of hrs after eating these foods symptoms start
How frequently these symptoms occur: Daily = 5, 2-3 times/week = 4,
Weekly = 3, Monthly = 2, Less often = 1, Not present = 0
Swelling of the lips, eyelids or tongue
Itchy, red, raised, burning, hot, nettle rash
Area affected by the rash: face, mouth neck, limbs body, all over -
continuous area = 1; 2 areas = 2; >3 areas = 3
Do new patches appear when old ones are disappearing? Yes = 2, No = 1
Do patches last more than 24 hr (2) or less than 24 hr (1)
Does urticaria come on with physical stimull cold, wet, wind, pressure..
How many years rash or swelling experienced
It antihistamines tried for urticaria were they effective? 0 = not tried 1 =
ineffective 2 = effective
Graded SPT 0 = neg, 1 =< hist 2 = ≧ hist HDM
Graded SPT 0 = neg, 1 =< hist 2 = ≧ hist Cat
Graded SPT 0 = neg, 1 =< hist 2 = ≧ hist Dog
Graded SPT 0 = neg, 1 =< hist 2 = ≧ hist Grass Pollens
Graded SPT 0 = neg, 1 =< hist 2 = ≧ hist Tree Pollens
Graded SPT 0 = neg, 1 =< hist 2 = ≧ hist Egg
Graded SPT 0 = neg, 1 =< hist 2 = ≧ hist Milk
Graded SPT 0 = neg, 1 =< hist 2 = ≧ hist Rice
Graded SPT 0 = neg, 1 =< hist 2 = ≧ hist Peanut
Graded SPT 0 = neg, 1 =< hist 2 = ≧ hist Hazelnut
Graded SPT 0 = neg, 1 =< hist 2 = ≧ hist Brazil nut
Graded SPT 0 = neg, 1 =< hist 2 = ≧ hist Almond
Graded SPT 0 = neg, 1 =< hist 2 = ≧ hist Walnut
Graded SPT 0 = neg, 1 =< hist 2 = ≧ hist to ANY nut
Graded SPT 0 = neg, 1 =< hist 2 = ≧ hist Latex
Total serum [IgE] in kU/l
Grade of Rast test to HDM
Grade of Rast test to Cat
Grade of Rast test to Dog
Grade of Rast test to Grass Pollens
Grade of Rast test to Tree pollens
Grade of Rast test to Egg
Grade of Rast test to Milk
Grade of Rast test to Soya
Grade of Rast test to Wheat
Grade of Rast test to Fish (cod)
Grade of Rast test to Rice
Grade of Rast test to Peanut
Grade of Rast test to Hazelnut
Grade of Rast test to Brazil nut
Grade of Rast test to Almond
Grade of Rast test to Walnut
Highest grade of RAST test to ANY nut
Grade of RAST test to Latex
Grade of RAST test to Apple
Grade of RAST test to some other fruit, vegetable or other food (not
specified in other columns) that is associated with symptoms
Grade of RAST test to any specific food (other than nuts) associated with
symptoms

At present there are no mathematical rules governing the required size of data subsets and most ANN-based studies utilize anecdotal rules derived from experience and analogy with statistical regression techniques (Basheer and Hajmeer et al 2000). Table 3 shows the allocation of the total (78 patients) data set into training and test data subsets. Data utilised for the ANN training subset for both parent databases were drawn from patients 001-057 since these were collected first and data from patients 058-078 were used as test data.

TABLE 3
Distribution of diagnoses for total patient data
set, training data subset and test data subset.

| | Total | Training | Test |
|---|---|---|---|
| Food intolerance | 27 | 20 | 7 |
| Multiple allergy | 15 | 10 | 5 |
| Nut allergy | 22 | 17 | 5 |
| Allergy to (an)other specific food(s) +/− nuts | 11 | 8 | 3 |
| Drug-induced GI symptoms | 3 | 2 | 1 |
| | 78 | 57 | 21 |

### Balancing of Training and Test Subset Data

It is desirable that data used in ANN training is nearly evenly distributed between output categories to prevent the ANN model generated from being biased to over-represented output classes (Swingler 1996). Table 3 shows the distribution of diagnoses amongst patients 001-057. Traditional approaches to dealing with such unbalanced data include removing examples from over-represented output classes or adding examples pertaining to under-represented classes (Basheer and Hajmeer 2000). The relatively small size of the training and test data subsets made the first option undesirable. Furthermore, whilst there is no published epidemiological data with which to compare the distribution of diagnoses in the training data subset, it seemed unlikely that significant numbers of under-represented diagnoses would be made. It was therefore decided to use unbalanced training and test data on the premise that models created would reflect what appeared to be a real-world bias to food allergy in patients presenting to the WCAS.

### Optimisation of ANN Architecture

The study used a commercially available ANN the Neuroshell Predictor™ (Ward Systems Inc, Frederick, Md., USA). Neuroshell Predictor™ can operate in one of two modes. In the neural mode of analysis, the neural net dynamically grows hidden neurons to build a model which generalises well and trains quickly. A variation of the Cascade Correlation algorithm is utilised. When applying the trained network to new data, the Neural Training Strategy may enable better results to be obtained on “noisy data” that is somewhat dissimilar from the data used to train the network.

Alternatively, the Neuroshell Predictor™ can be used in a genetic mode of analysis. A genetic algorithm is utilised, which is a variant of the General Regression Neural Network (GRNN). The Genetic Training Strategy trains slowly. When applying the trained network to new data, the Genetic Training Strategy gets better results when the new data is similar to the training data. It also works better when the training data is sparse.

### Neuroshell Predictor™ Data Output Format in Neural Analysis Mode

The Neuroshell Predictor™ could not initially be used to analyze the 79-input fields since the learning group size (57 patients) is smaller than the number of input fields. The genetic mode of analysis was applied initially, and later use was made of the neural mode of analysis after removing some of the input fields (i.e., on moving to models with a more limited number of inputs).

Subsequently, when the trained ANN operating in this mode was presented with the test data subset from patients 58-78, the results shown in Table 4 were obtained.

TABLE 4
Results obtained with ANN trained in genetic analysis mode, 79-input model.

| | Actual | | | | | | |
|---|---|---|---|---|---|---|---|
| | “Allergy to | “Drug-induced | [symbol] ual “Food | Actual “Multiple | Actual | | [symbol] ve |
| | a spec [symbol] | GI sym [symbol] | intoleran [symbol] | allergy” | “Nut allergy” | Total | Predictive |
| [symbol] assified as “Allergy to a | 2 | 0 | 0 | 0 | 0 | 2 | 100.00% |
| specific foo [symbol] | | | | | | | |
| [symbol] ssified as “Drug-induced GI | 0 | 1 | 0 | 1 | 0 | 2 | 50.00% |
| sympto [symbol] | | | | | | | |
| Classified as “Food intolerance” | 0 | 0 | 6 | 0 | 0 | 6 | 100.00% |
| Classified as “Multiple allergy” | 1 | 0 | 0 | 3 | 0 | 4 | 75.00% |
| Classified as “Nut allergy” | 0 | 0 | 1 | 1 | 5 | 7 | 71.43% |
| Total | 3 | 1 | 7 | 5 | 5 | 21 | |
| True-pos. ratio | 0.6667 | 1 | 0.8571 | 0.6 | 1 | | |
| False-pos. ratio | 0 | 0.05 | 0 | 0.0625 | 0.125 | | |
| True-neg. ratio | 1 | 0.95 | 1 | 0.9375 | 0.875 | | |
| False-neg. ratio | 0.3333 | 0 | 0.1429 | 0.4 | 0 | | |
| Sensitivity | 66.67% | 100.00% | 85.71% | 60.00% | 100.00% | | |
| Specificity | 100.00% | 95.00% | 100.00% | 93.75% | 87.50% | | |

[symbol]

indicates data missing or illegible when filed

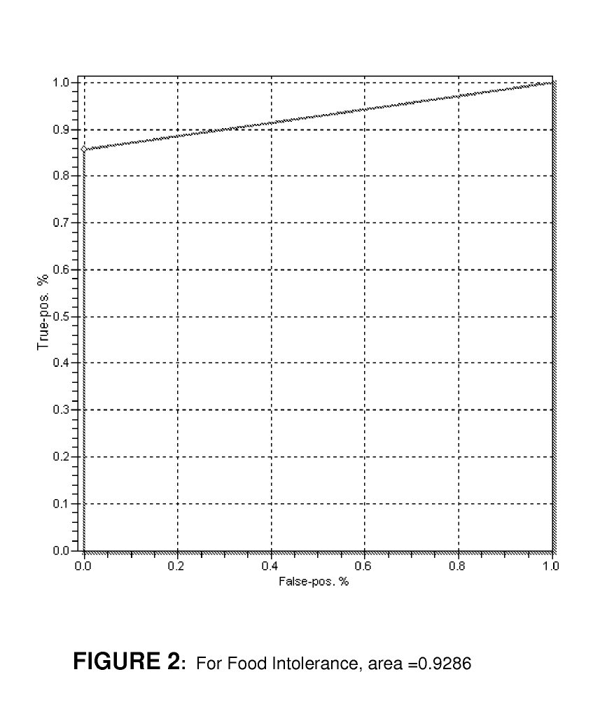

The associated Receiver Operating Characteristic (ROC) curve for this data is shown in FIG. 2 for the more common category of reactivity to food, which was food intolerance.

TABLE 5
Sensitivities and Specificities as function of the number of inputs for different training regimes.

| | | | Genetic - minimising total [symbol] | | Genetic: minimising Av % | |
|---|---|---|---|---|---|---|
| | | | incorrect classifications | | of incorrect classifications | |
| No of inputs | Neural Sensitivity | [symbol] ural Specific [symbol] | Sensitivity of [symbol] | Specificity [symbol] | Sensitivity of minim [symbol] | Specificity of minimising |
| 79 | | | 69.84% | 94.78% | 66.51% | 93.89% |
| 59 | | | 78.18% | 96.12% | 61.91% | 92.62% |
| 37 | 61.51% | 95.46% | 83.73% | 96.81% | 78.18% | 95.86% |
| 30 | 56.35% | 94.49% | 78.17% | 95.77% | 83.73% | 97.05% |
| 19 | 64.29% | 93.68% | 83.73% | 96.92% | 78.18% | 95.65% |
| 15 | 58.73% | 92.97% | 69.45% | 96.92% | 67.06% | 94.10% |

[symbol]

indicates data missing or illegible when filed

Data Analysis with View to Optimising Data Input and Diagnosis

The number and combination of data inputs was progressively reduced and varied, respectively, with a view to determining a preferred number and nature of inputs for producing a reliable diagnosis. This process partly involved an analysis of the relative importance of inputs into the ANN, and also utilised clinical experience and judgement. Table 2 shows, in addition to the 79-input model, 60-, 47-, 40-, 32-, 27-, 22-, 19- and 13-input models obtained using 60, 47, 40, 32, 27, 22, 19 and 13 data inputs, respectively. Using each input model, and each mode of operation of the ANN, data was obtained concerning the ANN reliability of diagnosis vis a vis use of clinical analysis. Table 5 shows the mean sensitivities and specificities across all 5-output diagnostic categories as a function of the number of input fields utilised. The results are shown separately for the ANN trained in the neural mode of analysis (for the 48- to 13-input models only), in the genetic mode of analysis when trained to minimise the average number of incorrect classifications and in the genetic mode of analysis when trained to minimise the total number of incorrect classifications. Tables 6 to 8 show sensitivities and specificities across the 5-output diagnostic categories for the 19-input model for the differently trained ANNs. It can be seen that the reduced input sets of 19- to 60-inputs provide good or excellent categorisation of food allergy by aetiological cause. The 19-inputs were reduced to 13-inputs by eliminating the following 6-inputs from the analysis:
1. taking any drugs that can cause those symptoms complained of.
2. symptoms triggered by nuts other than peanuts.
3. symptoms triggered by a specific food other than a nut.
4. symptoms triggered by a fruit and vegetables.
5. graded skin-prick test result to any nut (0=neg, 1=<hist 2=>histamine control).
6. the highest grade of RAST test to any nut tested (peanut, hazelnut, almond, walnut, brazil nut).

However, it was found that removal of the 6-inputs resulted in a marked degradation of the ANN's performance, with sensitivities falling to 50 to 60%. From this it was inferred that a meaningful classification of food allergy by aetiological cause should utilise an input data set which includes these 6-inputs. Of course, other inputs may be included as well. The removal of other combinations of 6-inputs from the 19-input model did not result in such a marked degradation in ANN performance.

TABLE 6
Sensitivities and specificities for the 19-input model with ANN trained in neural mode of analysis.

| | |
|---|---|
| Type of patient | Test |
| No of patients | 23 |
| No of inputs | 20 |
| Type of analysis | Neural |

| | Actual | “Drug- | [symbol] tual | Actual | Actual | | [symbol] e |
|---|---|---|---|---|---|---|---|
| | “Allergy | induced | “Food | “Multiple | “Nut | | Predictive |
| | to a spec [symbol] | GI symp [symbol] | intoleran | allergy” | allergy” | Total | Value |
| [symbol] assified as “Allergy to | 2 | 0 | 0 | 0 | 0 | 2 | 100.00% |
| a specific foo [symbol] | | | | | | | |
| [symbol] ssified as “Drug-induced | 0 | 1 | 0 | 0 | 0 | 1 | 100.00% |
| GI sympto [symbol] | | | | | | | |
| Classified as “Food intolerance” | 0 | 0 | 6 | 0 | 0 | 6 | 100.00% |
| Classified as “Multiple allergy” | 1 | 0 | 0 | 5 | 0 | 6 | 83.33% |
| Classified as “Nut allergy” | 0 | 0 | 1 | 0 | 5 | 6 | 83.33% |
| Total | 3 | 1 | 7 | 5 | 5 | 21 | |
| True-pos. ratio | 0.6667 | 1 | 0.8571 | 1 | 1 | | |
| False-pos. ratio | 0 | 0 | 0 | 0.0625 | 0.0625 | | |
| True-neg. ratio | 1 | 1 | 1 | 0.9375 | 0.9375 | | |
| False-neg. ratio | 33.33% | 0.00% | 14.29% | 0.00% | 0.00% | | |
| Sensitivity | 66.67% | 100.00% | 85.71% | 100.00% | 100.00% | | |
| Specificity | 100.00% | 100.00% | 100.00% | 93.75% | 93.75% | | |
| Mean Sensitivity | 90.40% | | | | | | |

| | | | |
|---|---|---|---|
| Mean Specificity | 97.50% | ROC curves for Food | 0.9890 |
| | | Intolerance Area = | |

[symbol]

indicates data missing or illegible when filed

TABLE 7
Sensitivities and specificities for the 19-input model with ANN trained in genetic
mode of analysis minimising the total number of incorrect classifications.

| | |
|---|---|
| Type of patient | Test |
| No of patients | 21 |
| No of inputs | 20 |
| Type of analysis | Genetic - minimising the total number of incorrect classifications |

| | Actual | “Drug- | [symbol] ual | Actual | Actual | | [symbol] e |
|---|---|---|---|---|---|---|---|
| | “Allergy | induced | “Food | “Multiple | “Nut | | Predictive |
| | to a spec [symbol] | GI symp [symbol] | intoleran [symbol] | allergy” | allergy” | Total | Value |
| [symbol] assified as “Allergy to a | 2 | 0 | 0 | 0 | 0 | 2 | 100.00% |
| specific foo [symbol] | | | | | | | |
| [symbol] ssified as “Drug-induced | 0 | 1 | 0 | 0 | 0 | 1 | 100.00% |
| GI sympto [symbol] | | | | | | | |
| Classified as “Food intolerance” | 0 | 0 | 6 | 0 | 0 | 6 | 100.00% |
| Classified as “Multiple allergy” | 0 | 0 | 0 | 3 | 0 | 3 | 100.00% |
| Classified as “Nut allergy” | 0 | 0 | 1 | 2 | 5 | 8 | 62.50% |
| Total | 2 | 1 | 7 | 5 | 5 | 20 | |
| True-pos. ratio | 1 | 1 | 0.8571 | 0.6 | 1 | | |
| False-pos. ratio | 0 | 0 | 0 | 0 | 0.2 | | |
| True-neg. ratio | 1 | 1 | 1 | 1 | 0.8 | | |
| False-neg. ratio | 0.00% | 0.00% | 14.29% | 40.00% | 0.00% | | |
| Sensitivity | 100.00% | 100.00% | 85.71% | 60.00% | 100.00% | | |
| Specificity | 100.00% | 100.00% | 100.00% | 100.00% | 80.00% | | |
| Mean Sensitivity | 89.14% | | | | | | |

| | | | |
|---|---|---|---|
| Mean Specificity | 96.00% | ROC curve for Physical | 0.9286 |
| | | urticaria Area = | |

[symbol]

indicates data missing or illegible when filed

TABLE 8
Sensitivities and specificities for the 19-input model with ANN trained in genetic mode of
analysis minimising average percentage of incorrect classifications over all categories.

| | |
|---|---|
| Type of patient | Test |
| No of patients | 21 |
| No of inputs | 20 |
| Type of analysis | Genetic - minimising average percent of incorrect classifications over all categories |

| | Actual | “Drug- | [symbol] ual | Actual | Actual | | [symbol] e |
|---|---|---|---|---|---|---|---|
| | “Allergy | induced | “Food | “Multiple | “Nut | | Predictive |
| | to a spec [symbol] | GI sym [symbol] | intoleran | allergy” | allergy” | Total | Value |
| [symbol] assified as “Allergy to a | 2 | 0 | 0 | 0 | 0 | 2 | 100.00% |
| specific foo [symbol] | | | | | | | |
| [symbol] ssified as “Drug-induced | 0 | 1 | 0 | 0 | 0 | 1 | 100.00% |
| GI sympto [symbol] | | | | | | | |
| Classified as “Food intolerance” | 0 | 0 | 6 | 0 | 0 | 6 | 100.00% |
| Classified as “Multiple allergy” | 1 | 0 | 0 | 4 | 0 | 5 | 80.00% |
| Classified as “Nut allergy” | 0 | 0 | 1 | 1 | 5 | 7 | 71.43% |
| Total | 3 | 1 | 7 | 5 | 5 | 21 | |
| True-pos. ratio | 0.6667 | 1 | 0.8571 | 0.8 | 1 | | |
| False-pos. ratio | 0 | 0 | 0 | 0.0625 | 0.125 | | |
| True-neg. ratio | 1 | 1 | 1 | 0.9375 | 0.875 | | |
| False-neg. ratio | 33.33% | 0.00% | 14.29% | 20.00% | 0.00% | | |
| Sensitivity | 65.67% | 100.00% | 85.71% | 80.00% | 100.00% | | |
| Specificity | 100.00% | 100.00% | 100.00% | 93.75% | 87.50% | | |
| Mean Sensitivity | 86.48% | | | | | | |

| | | | |
|---|---|---|---|
| Mean Specificity | 86.25% | ROC curve for Idiopathic | 0.9286 |
| | | urticaria Area = | |

[symbol]

indicates data missing or illegible when filed

Other forms of neural network and trained methodologies might be employed. For example, it may be desirable to analyse the data in a series of steps. For instance, an initial, broad diagnosis might be provided, with more detailed classifications into specific aetiological causes being provided in one or more further steps. A different and/or differently trained neural network may be used for a subsequent step, and a different subset of questions and/or test results might be used in order to make the more refined diagnosis. As an example of such a further, more refined diagnosis, the “multiple allergy” category discussed above might be further classified into separate oral allergy syndrome without pollen allergy, oral allergy syndrome with pollen allergy, oral allergy syndrome without nut allergy and oral allergy syndrome with nut allergy cause categorisations.

### REFERENCES

Basheer, I, A and Hajmeer, M. (2000). ‘Artificial neural networks: fundamentals, computing, design and application’. J. Microbiol Methods 43: 3-31.
Baxt, W, G. (1995). ‘Application of Artificial Neural Networks to Clinical Medicine.’ Lancet 346: 1135-1138.
Callan, R. (1999). ‘The Essence of Neural Networks’. Hemel Hempstead: Prentice Hall Europe.
Das, A; Ben-Menachem, T; Cooper, G, S, et al. (2003). ‘Prediction of outcome in acute lower-gastrointestinal hemorrhage based on an artificial neural network: internal and external validation of a predictive model.’ Lancet 362: 1261-1266.
Dybowski, R and Gant, V (eds) (2001). ‘Clinical Applications of Artificial Neural Networks’. Cambridge: Cambridge University Press.
Hanley, J, A and McNeil, B, J. (1982). ‘The meaning and use of the Area under a Receiver Operating Characteristic (ROC) Curve.’ Radiology 143: 29-36.
Hecht-Nielsen, R. (1990). ‘Neurocomputing’. Massachusetts: Addison-Wesley.
Holgate, S, T and Broide, D. (2003). ‘New targets for allergic rhinitis—a disease of civilisation’. Nature Rev Drug Discovery 2: 1-12.
Lancashire, L, J; Mian, S; Rees, R, C, et al. (2003). ‘Preliminary artificial neural network analysis of SELDI mass spectrometry data for the classification of melanoma tissue’. Proceedings of the 17^{th} European Stimulation Multiconference.
Linneberg, A; Nielsen, N, H; Madsen, F, et al. (2000). ‘Increasing prevalence of specific IgE to aeroallergens in an adult population: two cross-sectional surveys 8 years apart: the Copenhagen Allergy Study’. J. Allergy and Clin Imm 106: 247-252.
McCulloh, W, S and Pitts, W. (1943). ‘A logical calculus of the ideas immanent in nervous activity.’ Bull. Math. Biophys 5: 115-133.
Metz, C, E. (1978). ‘Basic principles of ROC analysis.’ Semin. Nuc Med 8: 283-298.
Roadknight, C; Palmer-Brown, D and Al-Dabass, D. (1997). ‘Simulation of correlation activity pruning methods to enhance transparency of ANNs.’ Int. J. Simulation 4: 68-74.
Rosenblatt, F. (1958). ‘The perceptron: a probabilistic model for information storage and organisation in the brain.’ Psychol Rev 65: 386-408.
Rumelhart, D, E and McClelland, J, L. (1986). ‘Parallel distribution processing: Explorations in the microstructure of cognition: Volume 1: Foundations’. Cambridge USA: MIT Press.
Schalkoff, R, J. (1977). ‘Artificial Neural Networks’. New York: McGraw-Hill.
Swingler, K. (1996). ‘Applying Neural Networks: A Practical Guide’. New York: Academic Press
Thompson, R. A, Bird A. G (1983). How necessary are specific IgE antibody tests in allergy diagnosis? Lancet, 321, 169-173.
Wei, J, T; Zhang, Z; Barnhill S, D, et al. (1998). ‘Understanding artificial neural networks and exploring their potential applications for the practicing urologist’. Urology 52: 161-172.
Wide, L, Bennich H, Johansson SGO (1967). Diagnosis of allergy by an in vitro test for allergen antibodies. Lancet, 2:1105.
Zweig, M, H and Campbell, G. (1993). ‘Receiver-Operating Characteristic (ROC) plots: a fundamental evaluation tool in clinical medicine.’ Clin. Chem 39: 561-577.

1. A method for diagnosing food allergy including:
(a) asking a patient each of the following questions:
are any drugs which can cause the symptoms complained of being taken or have recently been taken;
are symptoms triggered by nuts other than peanut;
are symptoms triggered by a specific food other than a nut;
are symptoms triggered by fruit and vegetables;
(b) carrying out each of the following tests:
skin prick test to a plurality of nuts to determine if there is a reactivity to any one of them;
RAST test to a plurality of nuts in order to determine the highest quantitative result; and
(c) inputting the results of the questions and tests into a neural network that has been trained to diagnose food allergy, wherein the highest quantitative result from the RAST test to a plurality of nuts is inputted; and
(d) producing an output indicative of a food allergy.

2. A method according to claim 1 in which:
part (a) further includes asking the patient the following questions:
is tingling of the mouth or lips, swelling of the tongue, the inside of the mouth or throat, difficulty swallowing, or difficulty breathing experienced after foods;
are symptoms triggered by wheat;
are symptoms triggered by milk;
are symptoms triggered by peanut;
are symptoms triggered by shellfish;
the time elapsed since eating a food implicated with causing symptoms and the symptoms appearing;
how frequently the symptoms occur;
length of time that rash or swelling has been experienced;
and part (b) further includes carrying out the following tests; skin prick test to grass pollens;
RAST test to milk;
RAST test to fish;
one of more RAST tests any fruit, vegetable or other food (other than egg, milk, soya, wheat, fish, rice, peanut, hazelnut, brazil nut, almond, walnut or apple) associated with symptoms;
RAST test to any specific food other than nuts associated with the symptoms.

3. A method according to claim 1 in which part (b) further includes carrying out the following tests;
RAST test to grass pollens;
RAST test to fish;
RAST test to apple.

4. A method according to claim 1 in which:
part (a) further includes asking the patient the following questions:
is nausea, vomiting, abdominal pain or diarrhea experienced after foods;
is wheezing or a worsening of asthma or eczema experienced after eating foods;
are symptoms triggered by cheese;
what areas of the body are affected by a rash;
and part (b) further includes carrying out the following tests;
RAST test to cat.

5. A method according to claim 1 in which:
part (a) further includes asking the patient the following questions:
number of first degree relatives with asthma, rhinitis or eczema;
is a nettle rash experienced after foods;
and part (b) further includes carrying out the following tests:
skin prick test to hazelnut;
skin prick test to walnut;
RAST test to rice.

6. A method according to claim 1 in which:
part (a) further includes carrying out the following tests:
skin prick test to HDM;
skin prick test to peanut;
skin prick test to brazil nut;
skin prick test to almond;
RAST test to HDM
RAST test to dog;
RAST test to peanut;
RAST test to brazil nut.

7. A method according to claim 1 in which:
part (a) further includes asking the patient the following questions;
are headaches experienced after foods;
are symptoms triggered by aspirin, aspirin-containing drugs, orange juice, curry, or high aspirin content food;
are antihistamines effective;
and part (b) further includes carrying out the following tests:
skin prick test for dog;
RAST test to egg;
RAST test to almond;
RAST test to walnut.

8. A method according to claim 1 in which:
part (a) further includes asking the patient the following questions:
number of pack years smoked;
are symptoms triggered by egg;
are symptoms triggered by fish;
are symptoms triggered by unidentified food additives;
and part (b) further includes carrying out the following tests:
skin prick test to cat;
skin prick test to tree pollens;
skin prick test to egg; skin prick test to milk;
skin prick test to rice;
total serum (IgE) detected;
RAST test to tree pollens;
RAST test to soya;
RAST test to hazelnut.

9. A method according to claim 1 in which one or more of the tests involves the provision of a quantitative result relating to the amount of allergen-specific IgE antibodies present.

10. A method according to claim 1 in which food allergy is diagnosed according to aetiological cause.

11. A method according to claim 10 in which nut allergy can be diagnosed by the neural network.

12. A method according to claim 10 in which at least one of food intolerance, drug-induced multiple allergy (oral allergy syndrome, with or without pollen allergy and with or without nut allergy) and allergy to other foods can be diagnosed by the neural network.

13. A computer system or apparatus, configured to aid in the diagnosis of food allergy, including:
(a) a device for obtaining data relating to a patient, wherein the data includes the results of a combination of questions and tests according to claim 1;
(b) optionally, a device for storing the data in storage means of the computer system;
(c) a device for transferring the data to a neural network trained on samples of the data; and
(d) a device for extracting from the trained neural network an output, the output being an indicator for the diagnosis of food allergy.

14. A neural network to aid in the diagnosing of food allergy, the neural network including:
an input layer having a plurality of input nodes into which can be inputted data which include the results of an combination of questions and tests according to claim 1;
and an output layer for producing an output;
in which the neural network is trained on data relating to a group of patients in which food allergy is present, wherein the data includes said results of said combination of questions and tests according to claim 1, so that the neural network is configured to identify a pattern of data which corresponds to food allergy by the output layer producing an output indicative of the diagnosis of food allergy.

15. A method for training a neural network to aid in diagnosing food allergy including:
(a) obtaining data relating to a group of patients in which food allergy is known, wherein the data include a combination of the results of the questions and tests according to claim 1;
(b) training a neural network to identify a pattern of data which corresponds to food allergy; and
(c) storing the neural network in storage means of a computer or on a computer-readable medium.

16. A computer program product including: a computer usable medium having computer readable program code and computer readable system code embodied on said medium for aiding in the diagnosis of food allergy, said computer program product including:
computer program code means, when the program code is loaded, to make the computer execute a procedure to:
(a) obtain data relating to a patient, wherein the data include the results of a combination of questions and tests according to claim 1:
(b) optionally, store the data;
(c) transfer the data to a neural network trained on the aforementioned data; and
(d) extract from the trained neural network an output, the output being an indicator for the diagnosis of food allergy.

17. A computer system including a first means for:
(a) obtaining data relating to a patient, wherein the data include the results of a combination of questions and tests according to claim 1; and
a second remote means, wherein said second means includes means for:
(b) optionally, storing the data;
(c) transferring the data to a neural network trained on the aforementioned data; and
(d) extracting from the trained neural network on output, the output being an indicator for the diagnosis of food allergy.

18-19. (canceled)
